(19) United States
(12) Patent Application Publication
(10) Pub. No.: US 2008/0045960 A1
(43) Pub. Date: Feb. 21, 2008

(54) LOCKING TPO PLATE AND METHOD OF USE

(76) Inventors: Kenneth BRUECKER, Ventura, CA (US); Alex Khowaylo, Waldwick, NJ (US); Michael Khowaylo, Mahwah, NJ (US); Gary Rhau, Morgantown, PA (US); Robert Young, Loganville, GA (US); Patrick White, West Chester, PA (US)

Correspondence Address: MOETTELI & ASSOCIATES SARL, ST. LEONHARDSTRASSE 4, ST. GALLEN, CH-9000, CH

(21) Appl. No.: 11/538,604

(22) Filed: Oct. 4, 2006

Related U.S. Application Data
(63) Continuation-in-part of application No. 10/809,034, filed on Mar. 25, 2004.

(51) Int. Cl. A61B 17/80

(52) U.S. Cl. 606/69; 606/60

(57) ABSTRACT

A TPO bone plate with an offset longitudinal axis has a bone-contacting bottom side and a top side. Sets of overlapping holes communicate through the plate from the top to the bottom side. The overlapping holes have multifaceted surfaces such as a threaded surface or a coaxial series of annular grooves. The sets of overlapping holes are adapted to receive a bone screw with a head and a bone-engaging thread.

### CROSS REFERENCE TO RELATED APPLICATIONS

This application is a continuation-in-part application of prior U.S. patent application Ser. No 10/809,034, filed on Mar. 25, 2004, the contents of which are incorporated herein by reference thereto and relied upon.

### BACKGROUND OF THE INVENTION

This invention relates to devices, implants and prostheses used in orthopedic surgery, and, more particularly, to bone plates used in Triple Pelvic Osteotomy (TPO), to join reformed bone and thus promote healing.

Bone plates have been used to repair fractured or opened bones at least since the time of the Incas. The innovations in this field have involved plate materials, plate form, and the means of fixing the plate across the bone fragments to be joined.

In an effort to deal with the fixation of a bone plate, a compressive screw system, also known as the DCS bone plate system, was developed and in use in trauma surgery for many years. The procedures for use of this system are well documented by the AO Institute, an institute having as one of its goals, the promotion of new orthopedic surgical procedures. This, system included a bone plate having slots communicating there through. A land in which the slot is wider at one end defines a stepped surface adjacent the portion of the slot that extends through the bone plate. The stepped surface is generally Cut with a spherical endmill, thus creating a spherical stepped surface.

In a still further developments bone plates have been developed having individual threaded apertures and non-threaded apertures interspersed along the length of the plate. In this and other designs, the distance between holes has become a standard. Although an improvement over the inserts noted above, the locking positions are pre-defined, and only available in limited locations, which also reduce surgical flexibility.

In yet another solution. PCT application no. WO01/54601 combines the features of the DCS system discussed above with a locking screw. This design combines the features of the DCS system with a locking screw. Such a system is known as the combi-slot. In this design, the stepped surface of the slot is generally ramped or tapered so as to be deeper at one end than at another. This enables the positioning and selective fixing of the bone plate for compressing two bone fragments together with a preload created by Wedging action. In this manner, the bones are placed in a position that the surgeon believes would best promote healing.

While patent application no. WO01/54601 has proven advantageous because screws can be locked to the plate, the presence of an unthreaded slot limits the users ability to have multiple orientations for the screw.

In a further development, the AO Institute has studied and proposed the use of endpegs which are rigidly fixed in the extreme ends of the bone plate. Such an arrangement has been shown to better resist the flexing of the bone than use of a bone screw alone. Flexing can otherwise loosen the connection between the bone plate and bone in other bone plate systems.

In another development. German patent DE 4341980 A1, published on Jun. 14, 1953 describes a bone plate 2 having, an elongated slot 8 in which the sidewalk of the long sides of the slot are not parallel and are further provided with an internal thread 9. Corresponding bone screws 3 or inserts 6 have a head 5 with an external taper 4 and thus can be fixed into any point along the length, but to various depths of penetration. Therefore, the final configuration upon fixing is indeterminate and, due to the small amount of contact between the threads of the insert or screw and the slot, as well as the fact that the screw will be able to slide in one direction, the design does not appear to lend itself to reliable fixing.

U.S. Pat. No. 5,324,290 shows a complex hone plate having slots with countersunk circular recessed cut at intervals along the slot (a similar arrangement is shown in U.S. Pat. No. 4,696,290). It further shows the bone plate torqued against the bone so as to at least marginally, conform to the shape of the bone (see FIG. 2). Other patents of interest include U.S. Pat. Nos. 3,716,050, 3,659,595, 5,681,311, 5,261,910, and 5,364,399, all showing combinations of conventional slots and recesses which do not fully accommodate a bone screw having a threaded head.

In a Triple Pelvic Osteotomy, it is necessary to treat a subluxed hip in a canine, which is a genetic abnormality. This is when the femoral head is not sufficiently covered (less than 50%,% coverage) by the rim of the acetabulum (see pg. 472 of Appendix A attached and incorporated herein by reference thereto).

Consequently, a TPO plate is made up essentially of two plates that are non-parallel to each other, being rotated with respect to each other about an axis, and fixed together by an offset web. Different cases necessitate different angular changes to best cover the femoral head. If a femoral head is covered 30 degrees instead of 50, for example, one would need to use a 20 degree TPO plate to reach 50,% coverage, and so on. The plate positions the bone al: the correct anatomical angle.

In another product variation, expandable, lockable inserts enter into the slots of a standard borne plate. When the bone screw passes through one of these inserts and is torqued down, the insert expands and locks the screw in place. However, this insert is locked in a secondary operation. This is not desirable because this requires more operating room time and adds complexity to the procedure. Further, the inserts must be added in the specific location before the plate is fixed to the bone and cannot be subsequently Inserted. This limits the choice of placement during surgery if the need arises.

Also, the above insert design relies on a friction lock via contact between two surfaces. Friction locks are not reliable and come lose more easily than threaded locked holes. The result of such a design is inferior to that of the threaded plate and screw designs discussed below.

In prior art TPO plates; it is known that the bone screws can come lose, causing pain and/or requiring corrective surgery. What is needed therefore is a TPO plate which can be firmly fixed to the bone, in a manner to minimize the likelihood of loosening of the bone screws. In addition, what is needed is a TPO plate where the holes are located to achieve the best anatomical location of the screws in the bone.

What is needed is a bone plate that provides greater flexibility of choice to the surgeon, in a bone plate that has multiple orientations for the locking screw and thus, plate placement, while reliably and permanently Fixing the bone plate to the bone fragments, in any hole position. More specifically, what is needed is a bone plate that provides this choice of plate placement while reliably and permanently fixing the bone plate to the bone fragments, in any hole position.

What is needed is a bone plate with holes that create at least uni-directional compression.

### SUMMARY OF THE INVENTION

A TPO hone plate is provided having an offset longitudinal axis, a bone-contacting bottom side and a top side. Sets of overlapping holes communicate through the plate from the top to the bottom side. The overlapping holes have multifaceted surfaces such as a threaded surface or a coaxial series of annular grooves. The sets of overlapping holes are adapted to receive a bone screw with a head and a bone-engaging thread.

An object of the invention is to provide an orthopedic surgeon greater flexibility of choice in that a threaded peg providing secure fixing can be positioned at any interval along the bone plate, including at its extreme ends.

### BRIEF DESCRIPTION OF THE DRAWINGS

FIG. 1 is a perspective view of the bone plate of the invention.

FIG. 2 is a side view of the bone plate of FIG. 1.

FIG. 3 is a front view Of the bone plate of FIG. 1.

FIG. 4 is a top view of the bone plate of the invention

FIG. 5 is a top view of a kit of the invention.

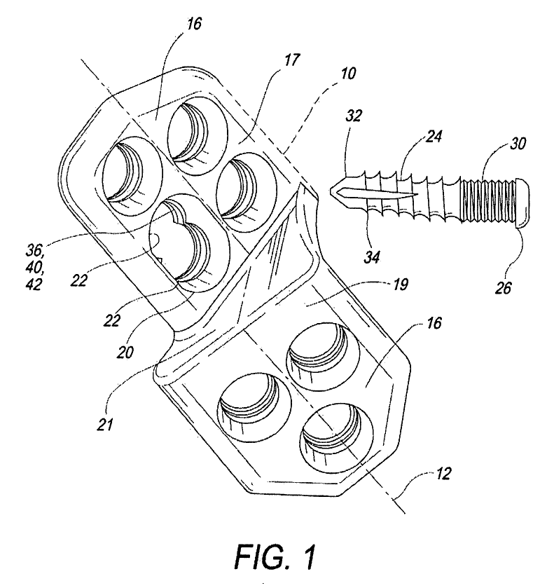

FIGs in Appendix A include figures that illustrate the use of the bone plate of FIG. 1.

### DETAILED DESCRIPTION OF THE PREFERRED EMBODIMENT

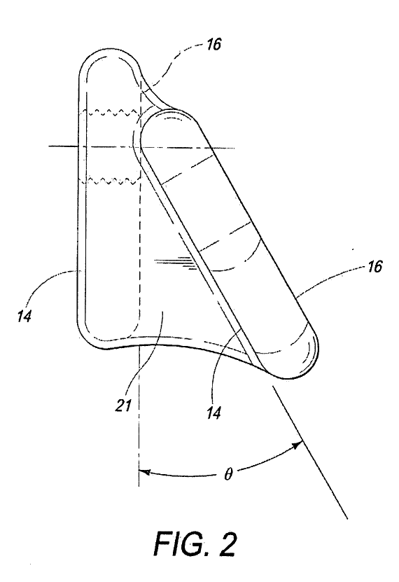

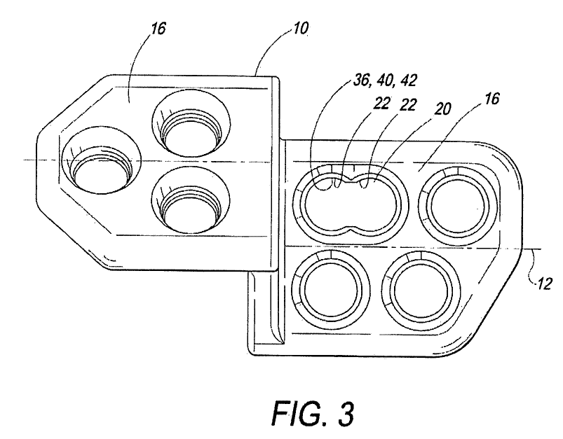

Referring now to FIGS. 1 to 3, a bone plate 10 with an offset longitudinal axis 12 has a bone-contracting bottom side 14 and a top side 16 comprised in two flanges 17 and 19 that are non-parallel to each other, being rotated with respect to each other about an axis by an amount θ, and fixed together by an offset web 21.

Sets 20 of overlapping holes 22 communicate through the plate 10 from the top side 16 to the bottom side 14. The overlapping holes 22 are adapted to receive a bone screw 24 with a head 26 having a thread 30 and, on an opposite end 32, a body having a bone-engaging thread 34.

The Sets 20 of overlapping holes 22 allow for further adjustability and flexibility in positioning of the bone plate 10 during surgery. The overlapping holes 22 are formed normal to the top side 16 of the plate 10.

The overlapping holes 22 have multifaceted surfaces 36. In one embodiment, the multifaceted surface 36 is a threaded surface 40. In another embodiment, the multi-faceted surface 36 is a coaxial series of annular grooves 42.

Overlapping holes 22 are formed individually at an angle Ø offset from normal to the top side 16 of the plate 10. Such allows further flexibility, of choice to the surgeon as to where and how to fasten the bone plate 10. Where these overlapping holes 22 are oriented perpendicularly to the top side 16 of the bone plate 10, he may chose to fasten the plates in a conventional manner, namely, perpendicular to the top side of the plate.

Because of the organic form of bones in canines, no two bones are identical. In fact among canines with hip abnormalities, variations from the norm can be very significant. Consequently, bone plates 10 must be provided to accommodate different angular changes in order to best cover the femoral head. If a femoral head is covered 30 degrees instead of 50, for example, one would need to use a 20 degree TPO plate to reach 50% r) coverage, and so oil. The TPO bone plate 10 therefore positions the bone at the correct anatomical angle.

In a preferred embodiment, some of the overlapping holes 22 are formed normal to the top side 16 of the plate 10.

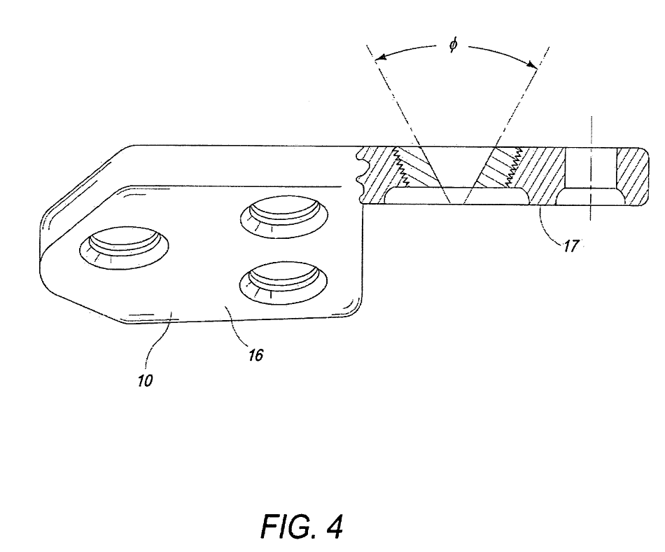

Referring now to FIG. 4, alternatively, some or all of the overlapping holes 22 may be formed at an angle Ø offset from normal to the top side 16 of the plate 10.

In an alternate embodiment, the bone plate 10 may include sets 20 of three overlapping holes 22 (not shown). Where these overlapping holes 22 are oriented perpendicularly to the top side 16 of the bone plate 10, the surgeon may chose to fasten the plates in a conventional manner.

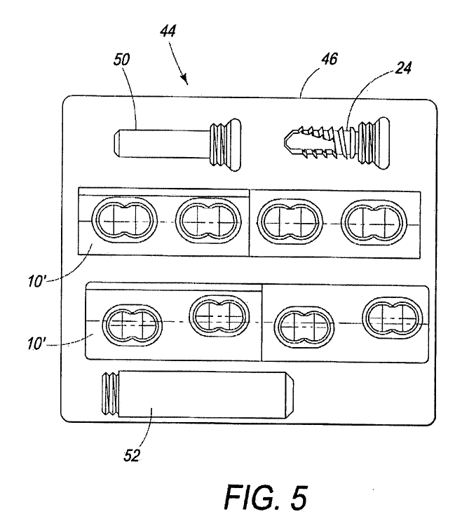

Referring now to FIG. 5, in another embodiment, orthopedic kits 44 are provided which include a case 46, a bone plate 10′, a variety of bone screws 24, threaded pegs 50 of various lengths, and a drill guide 52. The drill guide 52 has a threaded end 54 that threads into the thread 40 of an overlapping hole 22. The drill guide 52 has a main drill guide surface 56 to securely hold the drill guide in a desired orientation with respect to the bone plate 10 in order to stabilize a drill (not shown) used in an orthopedic procedure.

Note that the threaded apertures 100 used in the invention provide hole centers located at specific locations (as opposed to apertures that are formed as a slot). Use of threads centered at a specific point allows the bone screw to be fixed at a specific location at which the surgeon may judge the bone structure to be best suited to support such a bone screw. Unlike designs using a slot, the apertures 100 of the invention eliminate wander of the screw in the aperture. This further permits placement at specific locations for buttressing and/or secure fixing in neutral screw loading areas.

In a preferred embodiment, the bone plate 10 includes overlapping threaded holes 22 on one side of the web 21, thereby providing the ability to the surgeon of unidirectionally composing one bone fragment against the other.

In an alternate embodiment, shown in FIG. 4, the bone plate 10 includes overlapping threaded holes 22 on opposite sides of the web 21. This enables bi-directional compression of the bone fragments against each other to ensure more rapid healing.

A detailed description of the method of use of the bone plate 10 is attached as Appendix A, and incorporated herein by reference thereto.

In an advantage, the bone plate 10 provides greater flexibility of choice by providing multiple overlapping holes 22 oriented so as to maximize the surgeon's flexibility of placement of the plate.

In another advantage, the bone plate 10 uses locking screws which interface with corresponding threaded locking holes to better ensure secure fixing of the plate to the bone.

In still another advantage, the threaded apertures 40 of the bone plate 10 are provided with threads cut perpendicular to the top side 16 of the bone plate, as well as at an angle Ø to normal.

Multiple variations and modifications are possible in the embodiments of the invention described here. Although certain illustrative embodiments of the invention have been shown and described here, a wide range of modifications, changes, and substitutions is contemplated in the foregoing, disclosure. While the above description contains many specifics, these should not be construed as limitations on the scope of the invention, but rather as exemplifications of one or another preferred embodiment: thereof. In some instances, some features of the present invention may be employed without a corresponding use of the other features. Accordingly it is appropriate that the foregoing description be construed broadly and understood as being given by way of illustration and example only, the spirit and scope of the invention being limited only by the appended claims.

What is claimed is:

1. A TPO bone plate with an offset longitudinal axis, the bone plate comprising: two substantially flat flanges connected by a web, the flanges oriented non-parallel with respect to one another, the bone plate further having a bone-contacting bottom side and a top side with at least one set of overlapping holes which communicate through the plate from the top to the bottom side, wherein the at least one set of overlapping holes defines a threaded aperture having multifaceted surfaces.

2. The bone plate of claim 1, wherein the overlapping holes are formed normal to the top side of the plate.

3. The bone plate of claim 1, wherein the overlapping holes are formed at an angle offset from normal to the top side of the plate.

4. The bone plate of claim 1, wherein at least one of the overlapping holes is formed normal to the top side of the plate and at least a second of the overlapping holes is formed at an angle offset from normal to the top side of the plate.

5. The bone plate of claim 1, wherein the multi-faceted surface is a coaxial series of annular grooves.

6. The bone plate of claims 1, wherein the threaded aperture further comprises Sets of overlapping holes.

7. The bone plate of claim 6, wherein the overlapping holes are formed normal to the top side of the plate.

8. The bone plate of claim 6, wherein the overlapping holes are formed at an angle offset from normal to the top side of the plate.

9. The bone plate of claim 6, wherein at least one of the overlapping holes is formed normal to the top side of the plate and at least a second of the overlapping holes is formed at an angle offset from normal to the top side of the plate.

10. The bone plate of claim 6, wherein the sets of overlapping holes are aligned on the axis.

11. The bone plate of claim 6, wherein the sets of overlapping holes are positioned in a staggered arrangement from the longitudinal axis.

12. The bone plate of claim 11, wherein the overlapping holes are formed normal to the top side of the plate.

13. The bone plate of claim 11, wherein the overlapping holes are formed at an angle offset from normal to the top side of the plate.

14. The bone plate of claim 11, wherein at least one of the overlapping holes is formed normal to the top side of the plate and at least a second of the overlapping holes is formed at an angle offset from normal to the top side of the plate.

15. The bone plate of claim 1, wherein multi-faceted surface is a threaded surface.

16. The bone plate of claim 15, wherein the overlapping holes are formed normal to the top side of the plate.

17. The bone plate of claim 15, wherein the overlapping holes are formed at an angle offset from normal to the top side of the plate.

18. The bone plate of claim 15, wherein at least one of the overlapping holes is formed normal to the top side of the plate and at least a second of the overlapping holes is formed at an angle offset from normal to the top side of the plate.

19. The bone plate of claim 1 wherein the set of overlapping holes is adapted to receive a bone screw with a head and a bone-engaging thread.

20. The bone plate of claim 19, wherein the head of the bone screw has a plate engaging thread.

21. An orthopedic kit including:
a. a TPO bone plate with an offset longitudinal axis, a bone-contacting bottom side and a top side with at least one set of overlapping holes which communicate through the plate from the top to the bottom side, the overlapping holes defining a threaded aperture having multifaceted surfaces; and
b. at least one bone screw engagable with the bone plate.

22. The kit of claim 21, further comprising a drill guide having a main drill guide surface and opposite end portions, one end portion of which is securely engagable with the multi-faceted surface of a hole in the bone plate so as to securely hold the drill guide in a desired orientation with respect to the bone plate for stabilizing a drill used in an orthopedic procedure.

23. A TPO bone plate with an offset longitudinal axis, a bone-contacting bottom side and a top side with a plurality of sets of overlapping holes which communicate through the plate from the top to the bottom side, wherein the set of overlapping holes have threads adapted to receive a bone screw with a threaded head and a bone engaging threaded shank.

24. A TPO bone plate with an offset longitudinal axis, a bone-contacting bottom side and a top side with a plurality of sets of overlapping holes which communicate through the plate from the top to the bottom side, the overlapping holes having threaded surfaces adapted to receive bone screws with a threaded head and a bone engaging threaded shank, wherein the overlapping holes have centers substantially aligned along the longitudinal axis of the plate.

25. A TPO bone plate with an offset longitudinal axis, a bone-contacting bottom side and a top side with a plurality of threaded apertures communicating through the plate from the top to the bottom side, at least one of the threaded apertures comprised of overlapping holes having a threaded surface adapted to receive a bone screw with a head and a bone engaging thread, the overlapping holes further having centers staggered about the longitudinal axis of the plate.
